(19) United States
(12) Patent Application Publication
(10) Pub. No.: US 2005/0118593 A1
(43) Pub. Date: Jun. 2, 2005

(54) Method for detection and quantitative analysis of nucleic acid molecules comprising an extraction step with a strong base and applications

(76) Inventors: Gabrielle Potocki, Lautignac (FR); Laure Sabatier, Chatenay-Malbry (FR); Henri Benech, Paris (FR); Jacques Grassi, Bures sur Yvette (FR); Jean-Robert Deverre, Chatenay-Malabry (FR); Marie-Anne Marriere, Massy (FR)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/497,654

(22) PCT Filed: Nov. 14, 2002
(86) PCT No.: PCT/FR02/03889

(30) Foreign Application Priority Data
Dec. 7, 2001 (FR) 01/15843

(51) Int. Cl. C12Q 1/68

(52) U.S. Cl. 435/6

(57) ABSTRACT

The invention concerns a method for detection and quantitative analysis of nucleic acid molecules comprising a step which consists in extraction and random fractionation of said molecules with a strong base and applications thereof.

The present invention relates to a method for detection and quantitative analysis of nucleic acid molecules, comprising a step of extraction and of random fractionation of said nucleic acid molecules with a strong base, and to applications thereof.

Various methods for extracting nucleic acid molecules from cell suspensions are known. These methods generally comprise a step of cell lysis by enzymatic, osmotic or else chemical treatment, followed by a step for extracting the nucleic acid molecules, generally under alkaline conditions. According to the analyses that it is desired to carry out, there exist principally two main classes of extraction methods, namely extraction methods which make it possible to preserve the integrity of the nucleic acid (DNA) molecules of the cells intended to be analyzed and extraction methods which result in fractionation of the DNA.

Specifically, when it is a question of analyzing the effect of an outside factor, such as, for example, the effect of radiation or of a chemical or biological agent, it is essential for the extraction and analysis procedure not to cause further single-stranded or double-stranded cleavage of the DNA, so as not to decrease the sensitivity of the analytical method. More particularly, it is essential to properly control the alkaline conditions used, since they are often the cause of a cleavage background noise. These extraction and analysis techniques which make it possible to preserve the integrity of the DNA molecules are in particular described in the articles by DEAN et al., Nature, 1966, 209, 49-52; KOHN et al., Biochemistry, 1976, 15 (21), 4629-4637; OSTLING et al., Biochemical and Biophysical Research Communications, 1984, 123 (1), 291-298; SINGH et al., Experimental Cell Research, 1988, 175, 184-191 and BIHARI et al., Comp. Biochem. Physiol., 1992, 102B (2), 419-424.

However, these DNA extraction methods are often long, and comprise multiple limiting steps, in particular steps for isolating the DNA with respect to other biomolecules such as RNAs or proteins, which may be released in the course of the various treatment steps. These isolation steps are necessary in order not to disturb, or even prevent, the subsequent measurements.

Some authors have, however, recently developed a sensitive measurement of double-stranded DNA integrity, said measurement being carried out directly in a cell lysate using a new specific fluorochrome insensitive to the presence of RNAs, of single-stranded DNA or of urea in the lysate (BATEL et al., Analytical Biochemistry, 1999, 270, 195-200). These same authors also indicate that this quantification system no longer functions when the pH of the reaction medium exceeds 12.7.

However, for carrying out certain techniques for quantitative analysis of DNA, the presence of unfractionated DNA molecules is unsuitable. In fact, the viscosity and the size of long nucleic acid molecules are unsuitable or else require the use of denaturing or hybridization conditions which are not suitable for a given detection type.

Thus, in these particular cases, the DNA extraction methods must enable the DNA molecules to be fractionated.

With this aim, the use, for example, of enzymatic, radioactive or mechanical treatments has already been proposed. As an example of a fractionation method which is simple to carry out and which does not require dangerous manipulations, such as, for example, the use of X-rays, mention may in particular be made of the repeated pipetting several times of nucleic acid molecules extracted from cell lysates in order to obtain fragments of the order of 15 kilobases (kb) which are then used to detect telomeric lengths (NAKAMURA et al., Clinical Chemistry, 1999, 45, (10), 1718-1724).

However, this method of fractionation by successive pipetting is tedious, long and relatively unreproducible, and results in the production of DNA fragments which are sometimes still too long to be used in certain types of analyses. In addition, the method of DNA analysis used subsequent to this fractionation step results in only relative values being obtained.

Enzymatic DNA fractionation methods use restriction enzymes such as endonucleases which cleave DNA molecules at specific sites and thus result in restriction fragments having a specific sequence being obtained. It is therefore impossible to obtain random fragments of DNA by this technique.

The inventors have therefore developed the subject of the present invention in order to remedy all these drawbacks and to provide a method for quantitative analysis of DNA which is simple, rapid and reproducible and which has no limiting step.

The inventors have in particular demonstrated, surprisingly and unexpectedly, that it is possible to detect and to assay directly in a cell lysate random fragments of DNA obtained by treating cell suspensions with a strong base, under particular pH conditions.

A first subject of the invention is therefore a method for detection and quantitative analysis of nucleic acid molecules using a biological sample, characterized in that it comprises at least one step of extraction of said nucleic acid molecules, said extraction step comprising the following steps:
a) bringing a biological sample comprising said nucleic acid molecules into contact with a strong base having a molarity sufficient to confer on the biological sample a pH above 13,
b) lysing the mixture obtained until the biological sample has completely dissolved, and
c) neutralizing the reaction medium,
   so as to obtain neutralized random fragments of nucleic acid molecules.

This method has many advantages. It is simple, inexpensive, easy to carry out and completely reproducible. It does not use dangerous manipulations such as the use of X-rays. It can, in addition, be used on a large scale. Moreover, the random fragments obtained by carrying out this method have the advantage of being able to be directly used in methods for quantitative analysis of DNA, i.e. without an obligatory additional purification step.

According to the invention, the biological sample consists, for example, of cell pellet suspensions, of DNA purified from cell cultures or of tissue homogenates.

According to a first embodiment of the method in accordance with the invention, and when the biological sample used in step a) is a cell pellet suspension, then the cell concentration in said suspension is preferably between 5×10⁴ and 1×10⁸ cells/ml of strong base, and even more particularly between 5×10⁵ and 3×10⁷ cells/ml of strong base.

According to a second embodiment of the method in accordance with the invention, and when the biological sample used during step a) is DNA purified from cell cultures, then the DNA concentration is preferably between 3×10⁻⁴ and 0.6 mg of DNA/ml of strong base, and even more particularly between 3×10⁻³ and 0.3 mg of DNA/ml of strong base.

The nature of the strong base used in accordance with the invention is not critical. It can in particular be chosen from sodium hydroxide and potassium hydroxide, sodium hydroxide being particularly preferred.

The molarity of the strong base for bringing the pH of the biological sample to a pH value above 13 is generally between 0.7 and 1.5 M, a molarity of 1 M being most particularly preferred.

The lysis step b) is preferably carried out for a period of between 15 minutes and 24 hours. This contact time should, of course, be adapted to the nature of the biological sample to be analyzed.

In particular, when the biological sample consists of cell pellet suspensions or of tissue homogenates, the duration of the lysis step is preferably between 3 and 12 hours, so as to allow the sample to completely dissolve.

On the other hand, when the biological sample consists of purified DNA, the lysis period can be shorter, and is generally between 15 minutes and 2 hours.

Whatever its duration, the lysis step is preferably carried out at a temperature between ambient temperature and 100° C., and preferably between ambient temperature and 40° C.

When the biological sample consists of cell pellet suspensions or of tissue homogenates, the extraction step of the method in accordance with the invention can also comprise an additional step of sonication of the sample so as to accelerate the lysis step. This additional sonication step is preferably carried out after step a), within 15 to 30 minutes approximately following the bringing into contact of the sample with the strong base.

The sonication step can be carried out one or more times, in general once or twice, for periods ranging between 15 and 30 minutes.

The cell lysis step per se is then continued normally until the sample has completely dissolved.

This sonication step is not necessary when the sample consists of purified DNA.

The cell lysis is stopped by neutralization of the reaction medium.

During this neutralization, the pH of the reaction medium is then preferably brought to a value of between 6 and 8 approximately, and most preferably to a value of 7.5 approximately.

This neutralization step is preferably carried out by adding an acidifying agent or a buffer for lowering the pH of the reaction medium to the desired value.

According to a particular embodiment of the invention, this neutralization step is preferably carried out by adding 5 to 7 volumes of a buffer, and even more particularly by adding 6 volumes of a buffer, per volume of strong base. Six volumes of 1 M sodium phosphate buffer (pH 7) per volume of strong base are preferably used.

The neutralization is of course carried out after the lysis step b) and before the step for quantitative analysis of the DNA of the biological samples.

The method for detection and analysis in accordance with the invention can be used for determining, in absolute value, the amounts of DNA or the telomeric lengths of the DNA by techniques known to those skilled in the art, such as, for example, hybridometry (Chevrier et al., Mol. Cell Probes, 1993, 7, 187-197).

In addition, the method in accordance with the present invention can also be used for any application needing a cell count, for example, the screening of molecules intended to control telomeric length, which makes it possible to aid diagnosis in cancerology, to ensure the monitoring of premature aging, the monitoring of immunosenescence, the choice of treatments and therapeutic monitoring in chemotherapy, the development of research in the biology of aging; the counting of blood cells or the like in clinical examinations; the monitoring of stem cell extension in cell therapy and the measuring of the concentration of a drug such as antiretroviral agents in the context of HIV, etc.

The size of the random and neutralized fragments of nucleic acid molecules obtained using the method in accordance with the invention is generally between 50 and 50 000 base pairs (bp).

These random fragments can be directly used for the quantitative analysis of the DNA and cell counting.

A subject of the invention is therefore also the use of the neutralized random fragments in accordance with the invention, in a method for detection and quantitative analysis of DNA, and also for cell counting.

Besides the above arrangements, the invention also comprises other arrangements which will emerge from the following description, which refers to an example of extraction and quantification of DNA from cell extracts and of determination of the size of the telomers of chromosomes from eukaryotic cells, to an example of correlation between the amount of DNA and the peripheral blood mononuclear cell count when assaying intracellular metabolites, and to the attached figures in which:

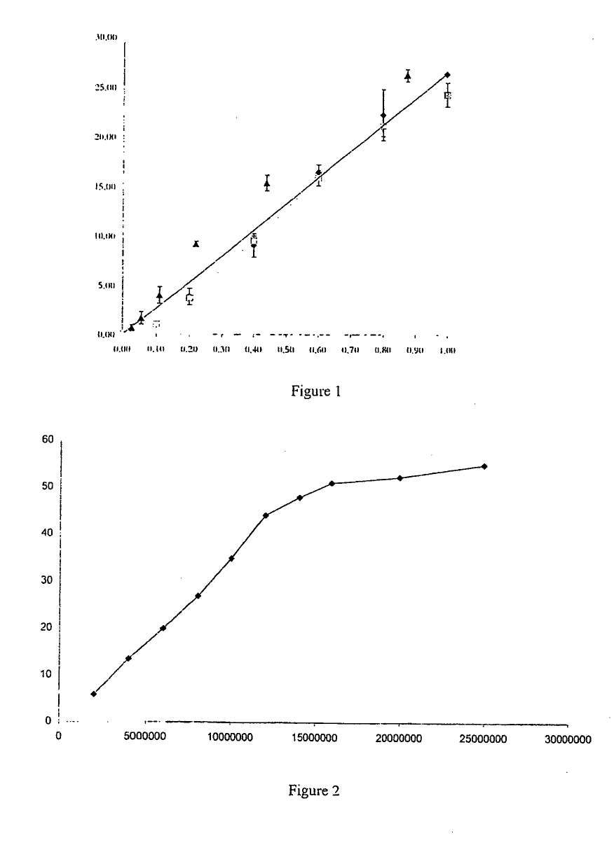

FIG. 1 represents the relationship between the fluorescence as a function of the amount of DNA, in the case of standard DNA (DNA) or of DNA extracted from diploid CIM cells (human fibroblast cultures);

FIG. 2 represents the relationship between fluorescence and the amount of cells extracted in 100 μl of 1 M NaOH, in the case of lysates diluted 10 times;

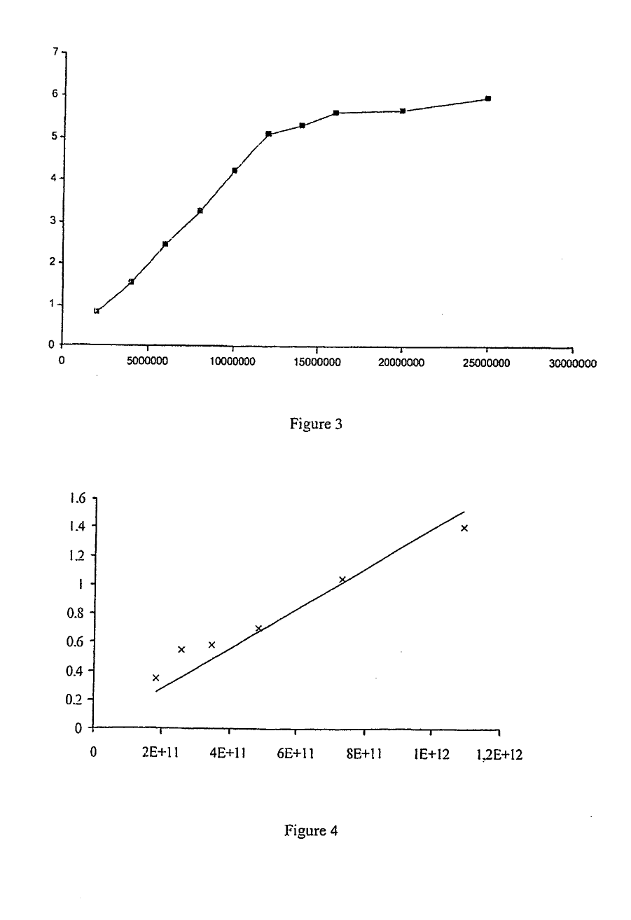

FIG. 3 represents the relationship between fluorescence and the amount of cells extracted in 100 μl of 1 M NaOH, in the case of lysates diluted 100 times;

FIG. 4 represents the values of absorbance at 414 nm as a function of the number of telomeric units/ml in the case of a cell lysate from CIM cells;

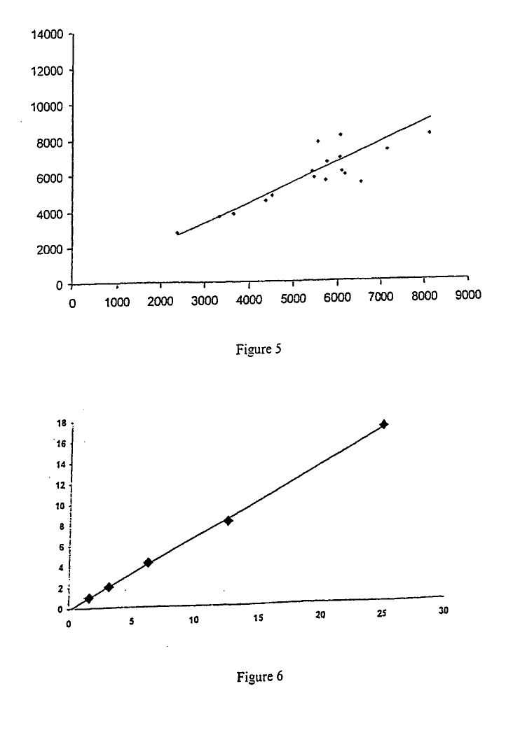

FIG. 5 represents the correlation between the mean telomeric lengths determined using the method in accordance with the invention (y-axis) and a determination carried out by the conventional Southern blotting method x-axis);

FIG. 6 represents the relationship existing between quantification of the number of cells on a Malassez cell x-axis) and the fluorescence signal obtained by the method for quantification of DNA according to the invention (y-axis).

### EXAMPLE 1

### Extraction and Quantification of the DNA from Cell Extracts and Determination of the Size of the Telomers of Chromosomes from Eukaryotic Cells

I—Materials and Methods

1) Preparation of Cell Lysates.

Eukaryotic cells from vertebrates (CIM human fibroblast cultures) are washed in phosphate buffered saline (PBS), and the pellets are then dried out, frozen in liquid nitrogen and stored at −80° C. or −20° C. for subsequent use.

After thawing at 4° C., the cells are taken up at 1×10⁶/100 μl of 1 M NaOH and vortexed. After 2×30 minutes of sonication at ambient temperature, the cell lysates are incubated at ambient temperature overnight, until the pellet has completely dissolved. The cell lysates are neutralized at pH 7.5 by adding 6 volumes of 1 M sodium phosphate buffer (pH 7) per volume of 1M NaOH.

2) Determination of the Size of the DNA Fragments Derived from the Cell Lysates

The lysates are partially desalted beforehand by diafiltration. After centrifugation of the samples (200 μl) at 13 000 g for 15 minutes and at 20° C., in Microcon®-30 units (Amicon), the retentates are diluted in 200 μl of water and then centrifuged again at 10 000 g for 5 minutes at 20° C. The retentates are recovered and loaded onto a new Microcon®-30, and then centrifuged at 10 000 g for 5 minutes at 20° C.

Thus, the samples (105 μl final volume) are diafiltered, partially desalted and concentrated 6.7 times.

The size of the DNA fragments can then be determined by 2% agarose gel electrophoresis.

3) Quantification of the DNA by Fluorescence

The cellular DNA fragmented during the extraction with sodium hydroxide is quantified by fluorescence using a Fluoroscan® II fluorimeter (Labsystems).

The sample consists of 10 μl of neutralized cell lysate or 10 μl of standard solution (DNA ladder XIV, Roche, at 250 ng/μl or DNA ladder 200 bp at 250 ng/μl, Roche) and 100 μl of SYBr Green reagent (Interchim, Molecular Probes, SYBR Gold nucleic acid, Ref. S11494) diluted extemporaneously to 1/10 000 in water. The linearity range for the method is between 0.1 and 10 ng of DNA.

Reading is performed after incubation for one hour at ambient temperature (between 20 and 25° C.), by excitation of the intercalating agent at 465 nm, and the emission reading is performed at 530 nm.

4) Determination of Telomere Length by Hybridometry According to the Invention

The method consists, firstly, in covalently binding a capture oligonucleotide complementary to the telomere sequences to a plate, for example a 96-well plate.

In vertebrates, the telomere sequence consists of a repetition of the unit TTAGGG. The capture oligonucleotide (SflA8) therefore has the sequence A8(TTAGGG)₆, to which the telomere sequences contained in the neutralized cell lysates bind by complementarity.

A tracer oligonucleotide (SflC8h) having the sequence (TTAGGG)₃ coupled to biotin in the 3′ position is then hybridized with the mixture. Detection is carried out by colorimetry, via coupling between streptavidin (which has affinity for biotin) and acetylcholinesterase, which allows the conversion of a colorless substrate, Ellman's reagent, to a colored product.

a) Covalent Binding of the Capture Oligonucleotide

The oligonucleotide SflA8 at 100 nmol/ml (1270 μg/ml) is incubated for 10 minutes at a temperature of 95° C., and then for 10 minutes in ice. The capture oligonucleotide (160 μl) is then diluted in a solution consisting of:
6 ml of a 100 mM 1-methylimidazole (1-MeIM) solution, pH 7 (prepared by diluting 398.6 μl of 1-MeIM in 220 μl of fuming HCl and a sufficient amount of water to have a final volume of 50 ml, said solution having been filtered through a 0.45 μm filter),
575.6 mg of 1-ethyl-3-(3-dimethylaminopropyl)-carbodiimide (EDAC), and
a sufficient amount of water to adjust the final volume to 59.84 ml.

The capture oligonucleotide is then incubated overnight at 50° C. in a 96-well plate comprising surface —NH functions (Covalent Binding NUNC®, Covalink), in a proportion of 100 μl per well. The oligonucleotide is therefore covalently bound to the plate by virtue of the terminal phosphate groups in the 5′ position of the capture oligonucleotide, via the amino groups, using the EDAC.

The plate is then washed 3 times with washing buffer (0.4M NaOH; 10 mM sodium dodecyl sulfate (SDS); preheated to 50° C., in a proportion of 200 μl per well, incubated for 5 minutes at 50° C., and then washed 2 more times.

The plate is then washed with deionized water and then stored at 4° C.

b) Assaying of Telomere Length

The oligonucleotide solutions, the standard lysates and the cell lysates are incubated for 10 minutes at 60° C. and then for 15 minutes at 4° C. on ice.

50 μl of tracer oligonucleotides SflC8h at 10 nmol/ml are diluted in 4950 μl of hybridization buffer (HB: 5 mM sodium phosphate buffer, pH 7.0, 0.75M NaCl, 5 mM EDTA, solution filtered through a 0.45 μm filter).

The non-neutralized cell lysates in 1M NaOH.

The plate is washed with distilled water. The wells are filled in a proportion of:
either 50 μl of tracer, 10 μl of standard lysate and 10 μl of 1M NaOH,
or 50 μl of tracer, 10 μl of non-neutralized cell lysate and 10 μl of HB buffer.

The plate is then incubated for 15 minutes at 40° C.

50 μl of 1M sodium phosphate buffer (pH 7) are then added to each well and the plate is then incubated overnight at 40° C.

The plate is washed twice with 150 μl/well of HB buffer.

100 μl/well of G4-Str (G4 form of streptavidin cholinesterase, AChE, EC 3.1.1.7 sold by the company SPI-BIO, Massy, France) at 1 EU/ml are then added before incubation of the plate for 2 hours at 20° C. The plate is washed twice with 150 μl/well of HB buffer.

200 μl/well of Ellman's reagent are then added and the plate is then incubated for between 3 and 24 hours at 20° C. in the dark.

The plate is then assayed by reading it at 414 nm.

To quantify the telomeric length, a relationship is established between the absorbance at 414 nm (A_{414 nm}) and the number of telomeric units/ml for a standard sample deposited onto the same plate as the samples to be assayed. This relationship corresponds to the following equation:
A₄₁₄ₙₘ=a×(telo units/ml)+b
in which a represents the slope of the line A₄₁₄ₙₘ=f (telomeric units/ml), and b the y-axis intercept point of this line, i.e. the absorbance measured when the number of telomeric units/ml=0.

It is considered that the 46 chromosomes of a diploid cell represent a mass of 6×10⁻¹² g. The number of telomere units of the standard sample is then calculated in the following way:
telo units/ml=46×C×L×2/(6×6×10⁻¹²),
with C=DNA concentration of the sample (g/ml) and L=length of a telomere in base pairs (bp).

As regards the sample to be assayed, the following calculation is established:
telo units/ml=(A₄₁₄ₙₘ−b)/a
chromosomes/ml=C×46/6×10⁻¹²
L(bp)=(telo units/ml)×6/[(chromosomes/ml)×2]
5) Determination of the Telomere Length by Southern Blotting Hybridization (Comparative Reference Method)

By way of comparison, the mean telomeric lengths were also measured by the conventional Southern blotting hybridization method.

To do this, the DNA is purified from 10⁶ cells by phenyl/chloroform extractions according to the methods commonly used by those skilled in the art. To estimate the size of the telomeric repeats, 2 μg of DNA sample are digested with RsaI and HinfI restriction endonucleases (Biolabs) at a concentration of 5 units/μg of DNA for 2 hours at a temperature of 37° C. The DNA sample thus digested is then separated by electrophoresis in a 0.6% agarose gel for 18 hours, and then transferred onto a nylon membrane and, finally, fixed on the membrane under ultraviolet radiation.

The membrane is then hybridized with a telomeric probe (TTAGGG)₅ labeled with γ-³²P-ATP using the random labeling kit sold by the company Roche.

The hybridization is carried out at 50° C. for at least 16 hours. The membrane is then washed twice with 2×SSPE buffer (0.2M phosphate buffer, 2.98M NaCl, 0.02M EDTA, pH approximately 7.4)-0.1% SDS and exposed overnight in a phosphorus detection cassette (Storm). The image acquisition is carried out using the NIH image V 1.6.2. software and the position of the size markers (1 kb ladder and λ-HindIII) is recorded on the image thus obtained. The migration distance is expressed in pixels and the intensity of the signal is expressed in grey levels. The densitometric profile of the telomeric fragments is then quantified using the Profit® program (Quantom Soft). After subtraction of the background noise, the mean telomere length is calculated according to the method described by C. Ducray et al., Oncogene, 1991, 18(29), 4211-4223.

II—Results

1) Determination of the Size of the DNA Fragments Obtained after Lysis and Sonication

After migration on agarose gel, the DNA, of which the size of the fragments is between less than 100 bp and more than 25 000 bp, is observed in the form of a smear.

(2) Quantification of the DNA

The results obtained appear in the attached FIG. 1, which represents the fluorescence (on the y-axis) as a function of the amount of DNA in ng, in the case of standard DNAs (1 kb ladder DNA, Roche (black diamonds) and 200 bp ladder, Roche (black squares)) or of DNA extracted from CIM cells (black triangles).

These results show that the variation in fluorescence is the same whether the DNA from a cell lysate or a standard DNA is involved.

The content of the neutralized cell lysates therefore has no influence on the assaying of the amount of DNA by fluorescence. The size of the fragments obtained does not influence the fluorescent signal obtained since the curves are identical for the cell lysates and the standard DNAs, which exhibit DNA fragments ranging in size from 11 000 bp to 10 bp.

The content of the neutralized cell lysates and the size of the DNA fragments obtained by lysing the cells therefore have no influence on the assaying of the amount of DNA by fluorescence.

FIGS. 2 and 3 in the appendix represent the amount of DNA extracted, assayed by fluorescence (on the y-axis), as a function of the number of cells in 100 μl of 1M NaOH (on the x-axis), in the case of neutralized lysates diluted 10 times (FIG. 2) and 100 times (FIG. 3).

These results show that, when the lysates are diluted 10 times, the relationship between the fluorescence and the number of cells is linear up to 12×10⁶ cells/100 μl of 1M NaOH; and when the lysates are diluted 100 times, the relationship is linear up to 13×10⁶ cells/100 μl of 1M NaOH.

This indicates that the loss of linearity is not due to the method of assaying by fluorescence used and described above, but to the lysis yield which decreases above 12×10⁶ cells/100 μl of 1M NaOH. Consequently, in order to obtain a constant lysis yield, use will preferably be made of cell pellets not exceeding 12×10⁶ cells/100 μl of 1M NaOH.

3) Determination of Telomere Length by Hybridometry According to the Invention

The results obtained are given in FIG. 4 in the appendix, which represent the values of absorbance at 414 nm (on the y-axis) as a function of the number of telomeric units/ml (on the x-axis), in the case of a CIM cell lysate.

4) Determination of Telomere Length According to the Southern Blotting Method and Correlation with the Results Obtained According to the Method in Accordance with the Invention

The telomeric lengths obtained using each of the two techniques described above (according to the invention and according to the Southern blotting method) are given in the appended FIG. 5.

In this figure, the mean telomere length (in base pairs) determined according to the method in accordance with the invention (y-axis) is compared to that obtained by the conventional Southern blotting method x-axis).

These results show a strong correlation between the two techniques for determining the mean telomere size (correlation coefficient: 0.75).

All these results demonstrate that the method for extraction and fractionation of DNA in accordance with the invention makes it possible to obtain a cell lysate which can be used in order to determine telomere length by hybridometry, more readily, more rapidly and more effectively than according to the techniques conventionally used in the prior art.

According to a particular embodiment of the invention, all the reagents used in this example can be provided in the form of a multicompartment device (kit) comprising at least one standard DNA solution, a capture oligonucleotide, a tracer oligonucleotide, a dilution buffer such as a potassium phosphate buffer, a strong base such as 1M sodium hydroxide, a fluorescent substrate such as SYBr Green, a hybridization buffer such as, for example, that described above, a streptavidin:enzyme complex such as the G4-Str described above, Ellman's reagent and one or more microtitration plates.

According to a particular embodiment of the this device, the oligonucleotide can be grafted onto the microtitration plates.

The kit as described above can be used during the method in accordance with the invention, to determine, in absolute value, the amounts of DNA or the telomeric length of the DNA in a biological sample.

### EXAMPLE 2

### Correlation Between the Amount of DNA and the PBMC Count when Assaying Intracellular Metabolites of NRTIs

In the context of intracellular assays for the phosphorylated metabolites of nucleoside reverse transcriptase inhibitors (NRTIs) for HIV in peripheral blood mononuclear cells (PBMCs), the number of PBMCs obtained for a sample after separation of the CPT tube is correlated with the amount of DNA labeled with a fluorescent intercalating agent.

I—Materials and Methods

1) Preparation of the Standard Range and of the Quality Controls

The PBMCs are isolated from three different blood bags provided by the Établissement Français du sang [French blood donation organization] (samples 1 to 3), using CPT tubes (Becton-Dickinson, ref. 332761) and according to the technique indicated by the manufacturer.

The number of isolated PBMCs is measured by counting with a Malassez cell. This counting is performed on two dilutions of the PBMC suspension (1/20 and 1/40), by depositing 20 μl of each dilution above and counting in duplicate on 10 squares.

A dilution A (standard 1) containing 25×10⁶ PBMCs is prepared. This dilution constitutes the most concentrated standard. It is then used to prepare the 6 standard calibration dilutions extemporaneously for each assay (cf. procedure point 3) below).

Using dilution A described above, several quality control (QC) series are also prepared, having the following concentrations:
QC No. 1=2×10⁶ PBMCs per sample
QC No. 2=5×10⁶ PBMCs per sample
QC No. 3=10×10⁶ PBMCs per sample
QC No. 4=15×10⁶ PBMCs per sample, and
QC No. 5=20×10⁶ PBMCs per sample.

Three QCs will be included in each analytical series, for example one QC No.1, one QC No.3 and one QC No.5.

Standard 1 and the QCs are then centrifuged at approximately 1200 g for 3 minutes at 4° C. After elimination of the supernatant, the standard and QC pellets are frozen dry at −80° C.

2) Sample Preparation

The samples derived from HIV-positive patients are prepared from a PBMC blood sample (approximately 8 ml) using CPT tubes as described above. The PBMC isolates are then centrifuged at approximately 1200 g for 3 minutes at 4° C. After elimination of the supernatant, the PBMC pellets are frozen dry at −80° C.

3) DNA Assay

This assay is carried on the standard 1, QC and sample pellets prepared above.

Procedure:

500 μl of a 0.05M Tris HCl, pH 7.4/methanol (30/70) mixture are added to each pellet. The content of the tube is then transferred into a new Eppendorf tube and centrifuged at 18 000 g for 30 minutes at 4° C. The supernatant is taken and stored for subsequent assaying of the NRTIs for HIV.

200 μl of 1M NaOH are added to the centrifugation pellet, and the pellet is then subjected to sonication for 30 minutes. The sonicated pellets are then incubated overnight at ambient temperature (between 20 and 24° C.).

The cell lysis is then stopped by adding 500 μl of 1M potassium phosphate buffer, pH 7.4.

The standard 1 cell lysate thus neutralized, containing 25×10⁶ cells, is serially diluted 2-fold in 1M potassium phosphate buffer, pH=7.4, in order to obtain the 6 calibration standards. The concentrations of the standards obtained are then as follows: 25×10⁶, 12.5×10⁶, 6.25×10⁶, 3.13×10⁶, 1.56×10⁶ and 0.78×10⁶ cells per standard.

The sample cell lysate (DNA extract) is diluted 1/10 in potassium phosphate buffer. 10 μl of DNA extract diluted 1/10 with 100 μl of SYBr Green (ref: S-7585, Molecular Probes) diluted 1/10 000 in millipore water are then deposited at the bottom of a well of an opaque microtitration plate (MUNCH®, Brand Product).

The reading is carried out after 60 minutes, using a Labsystems Fluoroscan® II fluorimeter (excitation 465 nm, emission 530 nm).

For the standards, the linear relationship between the results of the counts obtained on a Malassez cell and the proposed method for quantification of DNA is plotted. The regression line of type y=ax+b, where y is the value of the fluorescent signal and x the number of cells, is obtained (see appended FIG. 6).

In this figure, the result of the counts obtained on a Malassez cell is recorded on the x-axis (number of cells×10⁶) and the fluorescence signals obtained according to the method for quantification of the DNA in accordance with the invention are reported on the y-axis.

In this figure, y=0.6744x−0.0927 with a correlation coefficient (R²) of 0.9995.

For the samples and the QCs, knowing the value of y (fluorescence signal), the number of cells is calculated using this equation.

The values calculated for the samples should be included within the extent of the range.

For each sample, the measurement is carried out 4 times, each measurement taking place on different days.

II—Results

The results obtained are given in table I below:

TABLE 1

| | Fluorescence | | Difference with |
|---|---|---|---|
| | signal | Calculated | respect to the |
| | representative of | number of | value calculated |
| | the amount of DNA | cells | on a Malassez |
| Sample | in the sample | (10⁶ cells) | cell |
| Sample 1 | | | |
| Measurement 1 | 0.86 | 2.12 | 24.3 |
| Measurement 2 | 0.85 | 2.94 | −5.0 |
| Measurement 3 | 0.83 | 2.74 | 2.1 |
| Measurement 4 | 0.77 | 2.56 | 8.6 |
| Mean | | 2.59 | 7.50 |
| Standard | | 0.30 | |
| deviation | | | |
| CV (%) | | 11.7 | |
| Sample 2 | | | |
| Measurement 1 | 2.5 | 7.96 | 20.4 |
| Measurement 2 | 3.11 | 10.77 | −7.7 |
| Measurement 3 | 3.3 | 10.93 | −9.3 |
| Measurement 4 | 2.18 | 7.23 | 27.7 |
| Mean | | 9.22 | 7.78 |
| Standard | | 1.65 | |
| deviation | | | |
| CV (%) | | 17.9 | |
| Sample 3 | | | |
| Measurement 1 | 5.06 | 17.07 | 18.7 |
| Measurement 2 | 6.18 | 21.40 | −1.9 |
| Measurement 3 | 6.51 | 21.56 | −2.7 |
| Measurement 4 | 4.75 | 15.75 | 25.0 |
| Mean | | 18.95 | 9.79 |
| Standard | | 2.58 | |
| deviation | | | |
| CV (%) | | 13.6 | |

These results show that the method for quantitative analysis of nucleic acid molecules in accordance with the invention can be applied reproducibly to cell counting. In fact, the CV obtained is less than 20%, regardless of the initial quantity of cells (between 2.5×10⁶ and 19×10⁶ cells).

These results also demonstrate the accuracy of the method. In fact, when the results obtained using the method in accordance with the invention and the results obtained by conventional counting using a Malassez cell are compared, it is seen that the deviations between the values obtained by these two methods do not differ by more than 10%, regardless of the initial quantity of cells (between 2.5×10⁶ and 19×10⁶ cells).

According to a particular embodiment of the invention, all the reagents used in this example can be provided in the form of a multicompartment device (kit) comprising at least one calibration standard consisting of a known amount of PBMCs, a quality control consisting of a known amount of PBMCs, a lysed quality control consisting of a known amount of lysed PBMCs, a dilution buffer such as the potassium phosphate buffer, a strong base such as 1M sodium hydroxide, a fluorescent substrate such as SYBr Green and one or more microtitration plates.

The kit as described above can be used in the course of the method in accordance with the invention, for cell counting in a biological sample.

1-15. (canceled)

16. A method for detecting and quantitatively analyzing nucleic acid molecules using a biological sample, at least one step of which comprises extracting said nucleic acid molecules, comprising the steps of:
a) contacting a biological sample comprising said nucleic acid molecules with a strong base having a molarity sufficient to confer on the biological sample a pH above 13;
b) lysing the mixture obtained until the biological sample has completely dissolved; and
c) neutralizing the mixture:
thereby obtaining neutralized random fragments of nucleic acid molecules.

17. The method of claim 16, wherein the biological sample comprises cell pellet suspensions of DNA purified from cell cultures or of tissue homogenates.

18. The method of claim 16, wherein the sample is a cell pellet suspension and the cell concentration in the suspension is between 5×10⁴ and 1×10⁸ cells/ml of strong base.

19. The method of claim 16, wherein the biological sample is DNA purified from cell cultures and the DNA concentration is between 3×10⁴ and 0.6 mg of DNA/ml of strong base.

20. The method of claim 16, wherein the strong base is selected from the group consisting of sodium hydroxide and potassium hydroxide.

21. The method of claim 16, wherein the strong base has a molarity of between 0.7 and 1.5M.

22. The method of claim 16, wherein the lysis step b) is carried out for a period of between 15 minutes and 24 hours.

23. The method of claim 16, wherein the lysis step b) is carried out at a temperature between ambient temperature and 100° C.

24. The method of claim 16, further comprising sonicating the biological sample after step a) and within about 15-30 minutes after contacting the biological sample with the strong base.

25. The method of claim 16, wherein in the neutralization step c), the pH of the mixture is brought to between 6 and 8.

26. The method of claim 16, wherein the neutralization step c) is carried out by adding about 5 to 7 volumes of a buffer per volume of the strong base.

27. The method of claim 23, wherein the temperature is between ambient temperature and 40° C.

28. The method of claim 25, wherein the pH of the mixture is brought to about 7.5.

29. The method of claim 16, wherein said nucleic acid molecules detected and quantitatively analyzed have a size of between about 50 and 50,000 base pairs.

30. The method of claim 16, whereby absolute value amounts of DNA or telomeric lengths thereof or both in said biological sample are determined.

31. The method of claim 16, wherein said random fragments are not purified prior to said detecting and quantitative analyzing.

32. The method of claim 16, wherein the biological sample is purified DNA.

33. The method of claim 16, whereby cell counting in said biological sample is effected.

34. A multicompartment kit comprising at least one standard DNA solution, a capture oligonucleotide, a tracer oligonucleotide, a dilution buffer, a strong base, a fluorescent substrate, a hybridization buffer, a streptavidin:enzyme complex, Ellman's reagent and one or more microtitration plates.

35. A method of determining, in absolute value, amounts of DNA or telomeric lengths thereof or both in a biological sample, which comprises effecting the method of claim 16, using the kit of claim 34.

36. A multicompartment kit comprising at least one calibration standard comprising a known amount of PBMCs, a quality control comprising a known amount of PBMCs, a lysed quality of control consisting of a known amount of lysed PBMCs, a dilution buffer, a strong base, a fluorescent substrate and one or more microtitration plates.

37. A method of cell counting in a biological sample, which comprises effecting the method of claim 16, using the kit of claim 36.
